(19) United States
(12) Patent Application Publication
(10) Pub. No.: US 2024/0277269 A1
(43) Pub. Date: Aug. 22, 2024

(54) STATE MACHINE CALIBRATION

(71) Applicant: PercuSense, Inc., Valencia, CA (US)

(72) Inventor: Bradley Liang, Bloomfield Hills, MI (US)

(73) Assignee: PercuSense, Inc., Valencia, CA (US)

(21) Appl. No.: 18/395,313

(22) Filed: Dec. 22, 2023

Related U.S. Application Data
(60) Provisional application No. 63/446,272, filed on Feb. 16, 2023.

(51) Int. Cl. A61B 5/1495; A61B 5/145

(52) U.S. Cl. CPC A61B 5/1495; A61B 5/14532; A61B 2560/0223; A61B 2560/0266

(57) ABSTRACT

In one embodiment an analyte monitoring system is disclosed. The system includes a body worn device having a housing that contains a processor, a memory, a power supply and a sensor interface. The system further includes an analyte sensor that is coupled to the sensor interface. The analyte sensor has a proximal portion contained within the housing that is interfaced with the sensor interface. The analyte sensor also has a distal portion configured to be inserted into a host when positioned outside the housing. The system also includes a finite state machine that selects from possible sensor calibration coefficients based on a current state of the analyte monitoring system, an initial input, and longitudinal inputs received from the analyte sensor. Wherein the processor generates calibrated sensor values based on the selected calibration coefficients and longitudinal inputs from the analyte sensor.

### RELATED APPLICATIONS

This application claims the benefit of U.S. provisional application Ser. No. 63/446,272, filed on Feb. 16, 2023. The application listed above is hereby incorporated by reference in its entirety for all purposes.

### FIELD OF THE INVENTION

The present invention is generally directed to devices and methods that perform in vivo monitoring of an analyte or analytes such as, but not limited glucose, lactate or ketones. In particular, the devices and methods are for electrochemical sensors that provide information regarding the presence or amount of an analyte or analytes within a subject.

### BACKGROUND OF THE INVENTION

In vivo monitoring of particular analytes can be critically important to short-term and long-term well being. For example, the monitoring of glucose can be particularly important for people with diabetes in order to determine insulin or glucose requirements. In another example, the monitoring of lactate in postoperative patients can provide critical information regarding the detection and treatment of sepsis.

The need to perform continuous or near continuous analyte monitoring has resulted in the development of a variety of devices and methods. Some methods place electrochemical sensor devices designed to detect the desired analyte in blood vessels while other methods place the devices in subcutaneous or interstitial fluid.

Glucose sensors are one example of in vivo continuous analyte monitoring. Commercially available implantable glucose sensors generally employ electrodes fabricated on a planar substrate or wire electrodes. In either configuration the electrode surface is coated with an enzyme which is then further coated with a polymer membrane to control the amount of glucose and oxygen that reaches the electrode surface. In some glucose sensors the polymer membrane is hydrophilic which allows glucose to easily diffuse through the membrane layer, however the hydrophilic membrane severely limits the amount of oxygen that can diffuse through the membrane. The lack of oxygen on the electrode surface can become an issue because the glucose sensor works by using the enzyme to catalyze a reaction between glucose and oxygen resulting in hydrogen peroxide that is oxidized at a working electrode. Only when there is an abundance of oxygen present at the working electrode, will the glucose measured by the electrode be proportional to the amount of glucose that reacts with the enzyme. Otherwise, in instances where insufficient oxygen is present at the working electrode, the glucose measurement is proportional to the oxygen concentration rather than the glucose concentration.

An electrochemical reaction between the enzyme and analyte of interest generates an electrical current that can be detected and measured. In many embodiments, the electrical current generated by the reaction between the enzyme and analyte of interest is considered the raw signal. One challenge is correlating the raw signal generated by the electrochemical reaction with a glucose concentration within a subject. In many embodiments, the raw signal is adjusted using a calibration coefficient (or set of such coefficients) that associates or correlates the raw signal to a glucose concentration. Because of manufacturing variability, it may be difficult to determine a calibration coefficient for each individual sensor based on sensor design alone. Furthermore, it may be difficult to program or associate the determined calibration coefficient for a sensor throughout the manufacturing process including pairing and packaging the sensor with on-body electronics.

The claimed invention seeks to address many of the issues discussed above regarding applying calibration coefficients to a sensor system. In many examples discussed below, the analyte being measured is glucose. In still other examples the analyte is lactate. In still other embodiments combinations of two or more analytes are being measured. However, while specific embodiments and examples may be related to glucose or lactate, the scope of the disclosure and claims should not be construed to be limited to either glucose or lactate or glucose and lactate. Rather it should be recognized that the association of calibration coefficients may be applied to systems performing sensing of an analyte or analytes.

### BRIEF SUMMARY OF THE INVENTION

In one embodiment an analyte monitoring system is disclosed. The system includes a body worn device having a housing that contains a processor, a memory, a power supply and a sensor interface. The system further includes an analyte sensor that is coupled to the sensor interface. The analyte sensor has a proximal portion contained within the housing that is interfaced with the sensor interface. The analyte sensor also has a distal portion configured to be inserted into a host when positioned outside the housing. The system also includes a finite state machine that selects from possible sensor calibration coefficients based on a current state of the analyte monitoring system, an initial input, and longitudinal inputs received from the analyte sensor. Wherein the processor generates calibrated sensor values based on the selected calibration coefficients and longitudinal inputs from the analyte sensor.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1 is an exemplary block diagram illustrating components for a system to measure the presence or concentration of an analyte or molecule of interest, in accordance with embodiments of the present invention.

FIG. 2 is an illustration of exemplary initial operations or input that may be used as part of automatically applying or configuring a calibration factor for the sensor array, in accordance with embodiments of the present invention.

FIGS. 3A and 3B are exemplary illustrations of sensor states that may be transitioned through when implemented using a calibration state machine, in accordance with embodiments of the present invention.

FIG. 4 is an illustration of an exemplary hardware implementation of a calibration state machine, in accordance with embodiments of the present invention.

### DETAILED DESCRIPTION

Presented below are embodiments of a sensor system or analyte monitoring system that is intended to enable continuous real-time in-vivo electrochemical sensing of an analyte or molecule, or analytes or molecules of interest within a subject. The in-vivo measurement within a subject is typically performed in tissue such as, but not limited to subcutaneous tissue. However, various embodiments can be inserted into the vasculature, musculature, or organ tissue. The sensor may include a working electrode along with a counter electrode and a reference electrode. Alternatively, many embodiments utilize a working electrode in conjunction with a combined counter/reference electrode.

Embodiments of the analyte monitoring system can be configured to measure analytes such as lactate, ketones, glucose, tissue oxygen and the like. Furthermore, while some embodiments may be configured to measure a single or individual analyte, other embodiments can be configured to measure multiple analytes including various combinations of at least two or more molecules of interest such as lactate, ketone, glucose, oxygen, reactive oxygen species and the like. In still other embodiments, the sensors may be configured with infusion sets to enable sensing of a single or multiple molecule of interest while also enabling delivery of an infusate from a single point of entry.

Electrochemical sensors often rely on enzymes such as oxidase or dehydrogenase enzymes that are selected to react with the molecule of interest. Non-limiting examples include glucose oxidase to react with glucose, lactate oxidase to react with lactate, and 3-hydroxybutyrate dehydrogenase (3HBDH) to react with ketones. In the presence of the analyte of interest, the selected enzyme typically eventually generates hydrogen peroxide, NADP(H) or NAD(H) that is subsequently decomposed on the working electrode and the resultant electrical current is detected via some combination of a counter electrode, reference electrode, or combined counter/reference electrode.

In preferred embodiments, the generation/measurement of electrical current generated by the enzymatic reaction maintains a linear relationship with the concentration of the analyte of interest in the subject. Correlating the electrical current generated by the enzymatic reaction to the concentration of the analyte is a critical operation in order to generate actionable data. However, variability in manufacturing associated with parameters such as enzyme activity and fabrication tolerances can result in slight variations in sensor sensitivity and performance. Accordingly, it may be beneficial to include the capacity to determine or assign a calibration coefficient for each individual analyte being monitored. Moreover, it may be of further interest to enable modification of the calibration coefficient for each analyte being monitored based on real time data from the sensor system or analyte monitoring system.

The various embodiments discussed below are intended to be exemplary and should not be viewed or construed as discrete individual embodiments. Rather, where possible, individual features or elements discussed in each embodiment should be considered transferable or combinable with the various other embodiments disclosed below.

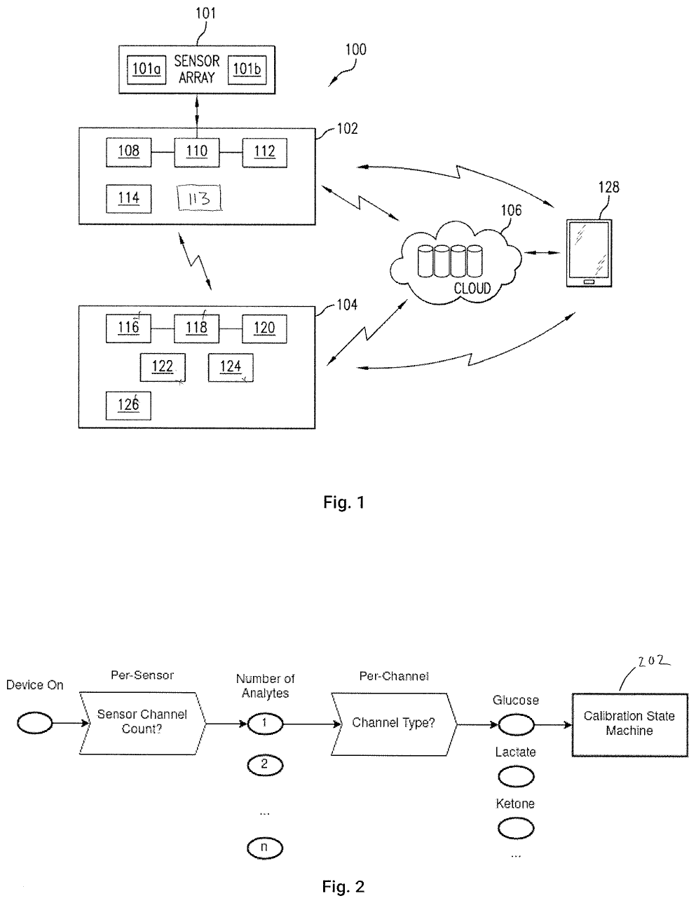

FIG. 1 is an exemplary block diagram illustrating components for a system 100 to measure the presence or concentration of an analyte or molecule of interest, in accordance with embodiments of the present invention. Broadly, the system 100 includes a sensor array 101 that includes at least a first analyte sensor 101a along with optional second analyte sensor 101b that are connected to an electronics module 102 that further enables bi-directional communication with at least one of a plurality of remote devices, such as, but not limited to an external monitor 104, cloud computing systems 106 and mobile devices 128. The disclosure of the first analyte sensor 101a and the second analyte sensor 101b should not be construed as limiting as the sensor array 101 may be configured to detect or measure additional analytes.

For example, in some embodiments the sensor array 101 may be configured to measure one, two, three or more analytes. In many embodiments, an analyte or analytes are measured or detected electrochemically. Exemplary analytes include, but are not limited to glucose, lactate, ketones, tissue oxygen, and the like.

The remote devices enable different aspects of functionality of the system 100, such as, but not limited to entry of subject specific data, display of historical and trending data acquired by the system 100, and machine learning. The totality of components illustrated in FIG. 1 enable the system 100 to be used across a variety of environments such as triage, subject monitoring and remote monitoring. However, embodiments tailored for a specific environment may not include all of the components shown in FIG. 1. For example, use of the system 100 as a remote monitor in the field may not utilize an external monitor 104. Likewise, when the system 100 is used as a monitor for multiple subjects, the system may not include a mobile device 128. The inclusion of all of the components in FIG. 1 is intended to illustrate the flexibility and adaptability of the system 100 to be used in different environments. However, regardless of environment, an element of the system 100 that is required for all embodiments is the sensor array 101.

The system 100 includes electronics module 102 that is capable of providing power for the sensor array 101 and may further enable bidirectional communication with other system components such as, but not limited to the external monitor 104, cloud computing systems 106 or mobile devices 128. Enabling the electronics module 102 to perform such tasks are components such as, but not limited to communication module 108, a processor 110, memory 112 and a power supply 114 enclosed within an electronics module case. The electronics module 102 includes additional components, however, the specific components included in FIG. 1 warrant discussion regarding operation of the system 100.

In preferred embodiments the power supply 114 provides power to the electronics module 102 and also to the sensor array 101. Batteries, rechargeable or disposable, can be used for the power supply 114. In order to minimize the likelihood of fluid ingress to the electronics module 102, it may be preferable to use inductive charging for embodiments using rechargeable batteries. Other embodiments use alternatives to batteries such as, but not limited to capacitors, supercapacitors, solar cells, fuel cells and the like. The specific examples provided for the power supply 114 should not be construed as limiting. Rather, the examples provided should be viewed as examples of portable power supplies capable of supplying the electronics module 102 and the sensor array 101 with power for the expected life of the system 100.

In some embodiments the processor 110 is a custom circuit such as, but not limited to an application specific integrated circuit (ASIC) or field programmable gate array (FPGA). In other embodiments the processor 110 is a more generic system on a chip (SoC) or system in package (SiP). In instances where an SoC or SiP is utilized, communication module 108 and memory 112 can be integrated within the SoC or SiP. In many embodiments the processor 110 is in communication with the sensor array 101 receiving raw signal data from the plurality of working electrodes. In some embodiments the processor 110 performs minimal manipulation of the raw data from the working electrodes. Examples of minimal manipulation include, but are not limited to, filtering noise and compression. In these embodiments the data from the working electrodes is transmitted to a multitude of external devices using the communication module 108 where processing of raw data from the working electrodes is completed. Alternatively, in other embodiments the processor 110 executes stored instructions to process the sensor data before transmitting processed data to any external devices via the communications module 108.

In many embodiments the communication module 108 is based on personal area network technology commonly referred to as Bluetooth low energy (BLE) or Bluetooth Smart. In other embodiments, a customized or semi-custom communication standard is utilized. However, one common trait for any communication module 108 is the ability to securely send and receive data between at least a third party device and the electronics communication module 102. The ability to securely transmit either raw or processed data using the communication module 102 enables the flexibility that allows the system 100 to be adaptable from a mobile monitor to being an integral component within a healthcare system.

In one embodiment data from the sensor array 101 is sent via the communications module 108 to a cloud computing system 1006, also commonly referred to as “the cloud”. In other embodiments data from the sensor array 101 is transmitted via the communications module 108 to an external monitor 104. Clinical settings such as a hospital ward where multiple monitors display a plurality of conditions being monitored for a subject may be ideal settings for embodiments where the electronics module 102 transmits to an external monitor 104 or the cloud 106. For example, with the appropriate infrastructure, data from the sensor array 101 can be transmitted in real-time to an electronic medical record stored in the cloud 106. Alternatively, in some embodiments data can be transmitted from the external monitor 104 to the cloud 106 where it is stored as part of an electronic medical record.

In still other embodiments, the electronics module 102 transmits data from the sensor array 101 to a mobile device 128 such as, but not limited to a smartphone, a smartwatch, a portable fitness monitor/tracker, a tablet, a notebooks computer, or an aftermarket or integrated infotainment center for a vehicle. The examples of a mobile device 128 are not intended to be construed as limiting. Rather, the examples are intended to provide guidance regarding the types of devices that can receive and/or transmit data to the electronics module 102. Accordingly, devices that can be viewed as similar to those listed should be considered contemplated by the current disclosure. In embodiments where the mobile device 128 includes a connection to the internet, the mobile device 128 can send data to the cloud 106 where the data can be archived, shared with other devices, be further processed or become data to enable machine learning. Utilizing the data to enable machine learning further enables data-driven improvements such as development of algorithms that are patient or area specific, or algorithms that are applied universally across all subjects. For example, depending on how much information is provided with the data provided for machine learning, subject specific algorithms can include, but are not limited to factors such as age, race, weight, and preexisting conditions. Similarly, regardless of subject specific information, all data processed via machine learning can be utilized to improve algorithms with the goal being improved outcomes for all subjects.

Even with embodiments where additional processing is handled on either an external monitor 104 or the cloud 106, memory 112 can be used to store data from the sensor array 101 on the electronic module 102. Using the memory 112 to store data from the sensor array 101 can ensure sensor data is not lost if there are connectivity interruptions between the electronics module 102 and the external monitor 104, the cloud 106 or a mobile device 128. The memory 112 can further be used to store program instructions for the processor 110, or to store values for variables used by the processor 110 to output sensor data.

In many embodiments the electronics module 102 is removably coupled with the sensor array 101. With these embodiments, the electronics module 102 is capable of being reused after the sensor array 101 is deemed consumed or depleted. In other embodiments, a permanent coupling is achieved after initial coupling between the electronics module 102 and the sensor array 101. In these embodiments, the electronics module 102 is considered disposable and is intended to be discarded after the sensor array 101 is deemed consumed. Alternatively, to reduce environmental impact, select portions of the electronics module, such as, but not limited to the power supply 114 and communications module 108 are recyclable. In many embodiments, initially coupling the electronics module 102 to the sensor array 101 provides power to the electrodes and initiates the program instructions stored in either the processor 110 or the memory 112.

In many of these embodiments, the electronics module 102 includes a feedback device 113. The feedback device 113 provides feedback regarding the status of the combined electronics module 102 and sensor array 101. For example, in some embodiments the feedback device 113 is a single or plurality of multicolored LEDs that blinks a first color and/or first pattern when the system is functioning within design parameters and a second color and/or second pattern if there is an error within the system. In other embodiments, the LED is a single color that uses different patterns of frequency of blinks to convey the status of the system. In still other embodiments, the feedback device 113 includes a vibration device similar to those used in mobile devices to convey the status of the system. In still other embodiments, a piezo or other audible sound emitting device is used as the feedback device 113.

The external monitor 104 may include some components not found in the electronics module 102, such as a graphic user interface (GUI) 122 and a display 124. Other components of the external monitor 104, such as a communications module 116, a processor 118, a memory 120 and a power supply 126 may seem duplicative of components in the electronics module 102, but may have different or improved capabilities or functionality. For example, while the power supply 114 of the electronics module 102 may be a battery, the power supply 126 for the external monitor 104 may include an alternating current power supply that is supplemented with a rechargeable battery to enable the external monitor 104 to operate seamlessly between being plugged into a wall socket and being moved as a portable device until it can be eventually plugged back into a wall socket.

For purposes of this invention, the GUI 122 further includes human interface devices that enable interaction with the GUI such as, but not limited to virtual or physical keyboards, touchscreens, joysticks, control pads and the like. Accordingly, use of the GUI 122 in conjunction with the display 124 enables user input to the system 100 and further allows selection or customization of what is shown on the display 124. The GUI 122 in conjunction with the communication module 116 and the communication module 108 further enables settings on the electronics module 102 to be manipulated or adjusted to optimize output from the system 100. Similarly, the GUI 122 enables user input to the processor 118 or the memory 120 to enable input and adjust settings that are applied to data from the sensor array 101.

The system further optionally includes a mobile device 128 having a user interface, such as, but not limited to a smartphone, a mobile phone, a smartwatch, a laptop, a tablet computing device, a pager and the like. The mobile device 128 is configured to receive data from the electronics module 102 via at least one of the cloud 106, the external monitor 104, or the electronics module 102. In many embodiments the mobile device 128 is in bidirectional communication with the electronics module 102 which enables input via the user interface of the mobile device 128 to be transmitted to the electronics module 102. This enables a user of the mobile device 128 to manipulate, configure, or program settings on the electronics module 102. In some embodiments, bidirectional communication enables processing of data from the sensor array 101 on the mobile device 128. Additionally, in embodiments where the mobile device 128 includes a display, real time data and trends derived from the data is shown on the mobile device 128. In embodiments where the mobile device 128 includes at least one of an audible, tactile and visual alarm, the mobile device 128 can be used to update users of the mobile device 128 of the status of the subject wearing the sensor array 101. The status of the subject includes, but is not limited to whether the system 100 is functioning properly, faults within the system 100, or real time measurements from the sensor array 101.

Another optional component within the system 100 is the cloud 106. Generally, the cloud 106 is considered some type of cloud computing which can be generalized as internet based computing that proves on demand shared computing processing resources and data to computers and other internet connected devices. In some embodiments the cloud 106 receives data from the electronics module 102 directly. In other embodiments data from the electronics module 102 is transmitted to the mobile device 128 before being transmitted to the cloud 106. In still other embodiments, the cloud 106 receives data from the electronics module 102 via the external monitor 104. In still other embodiments, various permutations of communications initiated by the electronics module 102 and transmitted between the external monitor 104, and the mobile device 128 results in data being transmitted to the cloud 106.

Data transmitted to the cloud 106 may have already been processed by an intermediary device or can be processed on the cloud 106 and transmitted back to the intermediary device. In some embodiments, the cloud 106 contains electronic medical records and data from the sensor array 101 is automatically uploaded to the electronic medical record. With real time data being uploaded to the cloud 106, it becomes possible to apply machine learning which can further enable automatic or semi-automatic adjustments to the electronics module 102. Automatic updating can result in changes to the programming of the electronic module 102 without human intervention whereas semi-automatic updating would require someone to confirm changes to the programming of the electronic module 102. In one example, the cloud 106 enables examination of medical history such as pre-existing conditions or exposure of subjects in close proximity to each other to inhibition agents and machine learning can suggest or set thresholds and sensor sampling rates based on previous data from subjects experiencing similar conditions and timing.

The previously discussed components or elements within the system 100 are intended to be exemplary rather than limiting. As the system 100 is intended to be flexible, components are able to be added or removed based on immediate needs. This includes enabling or disabling system components within one environment while enabling or disabling the same system components at a later point. For example, a facility utilizing the system from triage may not implement or enable communications to mobile devices 128 while enabling communication with the cloud 106. However, once a subject is moved from triage to a monitoring or remote monitoring environment, communication with a mobile device 128 may be enabled.

Furthermore, “sensor” or “sensor assembly” as used herein is any device, component or combination that (1) detects/records/communicates information about an event or the presence/absence of a particular analyte, thing or property in its sensing environment, and/or (2) indicates an absolute or relative value/quantity/concentration, or rate of change, of that analyte, thing or property.

The sensor may be based on any principle and can be an electrochemical sensor, an impedance sensor, an acoustic sensor, a radiation sensor, a flow sensor, an immunosensor, or the like. For in-vivo use in medical and veterinary applications, the sensor may be used to detect, measure and/or record (1) one or more analytes, such as, but not limited to glucose, lactate, oxygen, ketone, or any other marker(s) of a disease or medical condition, and (2) one or more of properties, such as temperature, pressure, perfusion rate, hydration or pH.

The use of the sensing devices described herein are also not limited to a specific physical structure of the sensor or infusion device. For example, in a glucose sensing application, the sensor may be similar to a conventional glucose sensors that use a glucose-limiting membrane and generally based on the principles of one-dimensional diffusion, where glucose and oxygen travel in the same general direction before reacting within the enzyme layer (e.g., glucose oxidase) at the working electrode. Or the sensor can use any other non-conventional structure, based on a glucose sensor without a glucose limiting membrane and/or any structure that takes advantage of multi-dimensional diffusion.

In many embodiments the system 100 described above may benefit from calibration of signals from the sensor array 101. Calibration of raw sensor signals from the sensor array 101 converts or transforms the raw signal data (e.g., electrical current generated by the electrochemical reaction occurring on each respective sensor) to values representative of the concentration of the analyte being measured or detected within a subject. In some embodiments sensor calibration is accomplished by applying a single calibration factor to raw sensor data. In other embodiments, sensor calibration can include multiple calibration factors that may be optionally applied based on real time data from the sensor array 101.

FIG. 2 is an illustration of exemplary initial operations or initial input that may be used as part of automatically applying or configuring a calibration factor for the sensor array 101, in accordance with embodiments of the present invention. In many embodiments the operations described below are intended to be executed on a state machine that may be implemented in software or hardware. Implementation of a state machine in hardware may be beneficial because it may remove or reduce dependency for on-device memory in generating calibrated sensor data. A reduction of on-device memory may lower or reduce overall cost and physical size of the system while further simplifying manufacturing. Another potential benefit of implementing the state machine in hardware is a reduction of a computational load on the device microprocessor. A reduction in computational load may reduce the power requirements of the overall system enabling smaller power supplies (e.g., batteries) and further enable reduction of physical size of the system. Additionally, reduction of the computational load may enable lower cost microprocessors thereby making the overall system more affordable. A further benefit of implementing calibration in a hardware based state machine is the separation of electronics associated with sensor measurements from electronics that may also be tasked with communications, display and reporting-related functions.

The operations in FIG. 2 begin with the sensor array being turned on. Once a connection to the sensor array is confirmed the system may identify an initial calibration state. An exemplary, non-limiting method or technique to identify an initial calibration state includes communicating a code wirelessly via a connected display or controller. Codes may be based on manufacturing lot information and could be entered manually or provided by other means such as, but not limited to scanning a bar code or quick response code. Another exemplary method to establish an initial calibration is the using a physical interface device between the electrical connection pads on the sensor or between the electrical connection pads on the sensor and the on-body electronics. For example, in some embodiments electrical connection pads on the sensor can be formed in specific patterns to convey an initial state. Alternatively, sacrificial pads or dialed in resistance between two pads may be used to convey or communicate the necessary information to establish an initial state.

In some embodiments the initial calibration state may include, but is not limited to states such as a number of sensor electrodes, how many analytes are being monitored or measured, and an analyte type for each sensing channel. Exemplary, non-limiting types of analytes include glucose, lactate, ketones and tissue oxygen. In other embodiments, other state information that may be included in the initial state includes a model type that relies on linear or logarithmic regression calibration factors.

After successfully determining the initial calibration state the device may determine multiple calibration states via individual calibration state machines 202 for each sensing channel. Accordingly, for embodiments with a single channel, a single calibration state machine 202 may determine a single calibration state for the single channel. For embodiments with multiple channels, multiple calibration state machines 202 may be employed in serial or parallel to determine a calibration state of each channel.

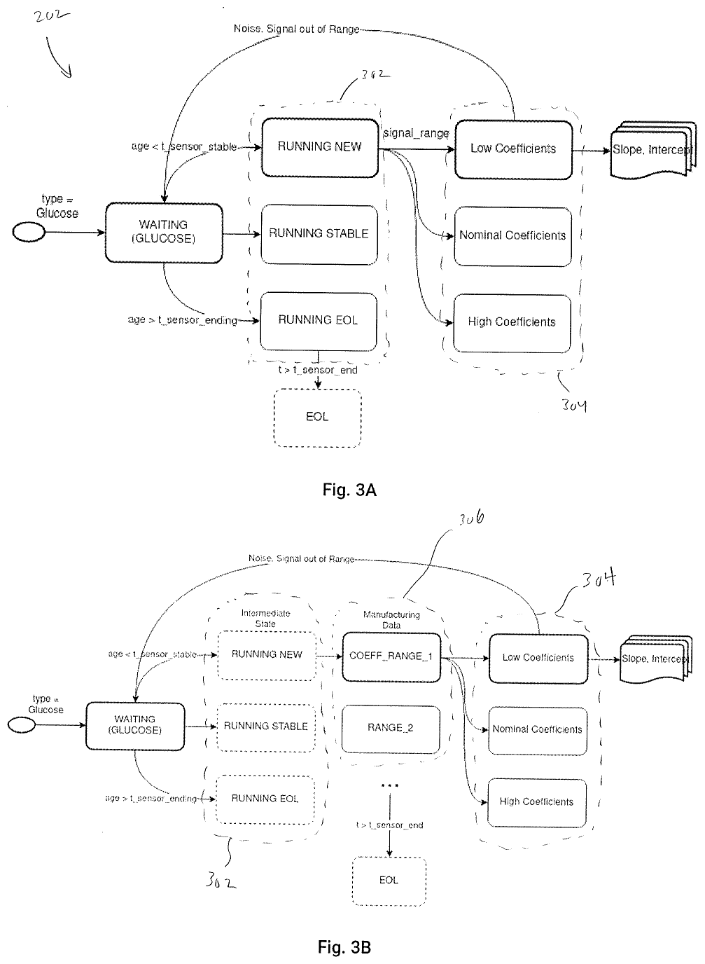

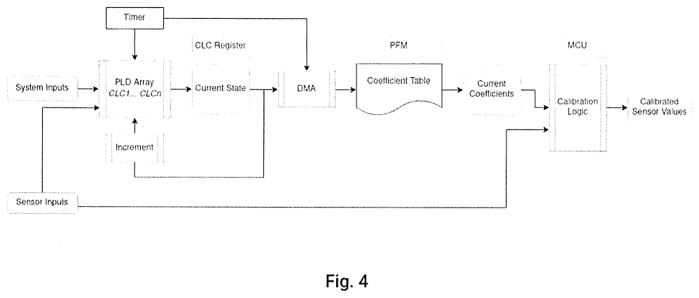

FIGS. 3A and 3B are exemplary illustrations of sensor states that may be transitioned through when implemented using a calibration state machine 202, in accordance with embodiments of the present invention. In many embodiments, the states that are included in FIGS. 3A and 3B are entered and transitioned through on a per-channel basis. In some embodiments, the state of one channel may also serve as an input into the state of another channel. FIG. 3A is representative of a single state path where each of the RUNNING states 302 and each of the COEFFICIENT states 304 could have their own child states. In FIGS. 3A and 3B, the calibration state machine 202 is defined by the type of sensing electrode being identified as type=Glucose. The exemplary state after identification is “WAITING”. In the many embodiments, in the WAITING state a device timer is triggered to begin providing periodic longitudinal input to the state machine. In many embodiments, longitudinal input may be also viewed or described as time-varying input. Exemplary, non-limiting longitudinal input includes, but is not limited to data such as sensor age (e.g., time elapsed since entering the WAITING state) along with sensor measurement values.

In many embodiments, if the sensor age and sensor measurement values (e.g., raw sensor data or current being generated by the sensor) meet certain defined conditions the device can transition to a new state illustrated collectively as RUNNING states 302. Non-limiting examples of conditions or state inputs that may enable transition from WAITING to RUNNING, include but are not limited to sensor age being within an acceptable range, sensor measurement values being within an acceptable range, change of sensor current being within an acceptable range and rate of change of sensor current being within an acceptable range. Another condition that may enable a transition between states is associated with noise level (e.g., variance in sensor current) being above or below a preferred threshold. In many embodiments, one or more condition or state input may be used to enable a transition between states. Transition to the RUNNING states 302 instructs the system to compute or determine calibration coefficients based on current state inputs. The calibration state machine 202 may remain in its current RUNNING state 302 until triggered by changes in periodic longitudinal state inputs.

In some embodiments some types of sensors may have an expected change in sensitivity over time. These expected changes may be compensated for by adjusting coefficients associated with conversion of the sensor current to a value representative of the analyte of interest. In many embodiments, expected or predictable changes in sensitivity of a sensor may result in changes to a coefficient commonly referred to as “sensitivity” or a “sensitivity shift”. In other embodiments, changes in sensor performance may result in adjusting or changing a coefficient commonly referred to as “offset” or an “offset shift”. For example, in an embodiment when the sensor age exceeds a predetermined threshold, the calibration state machine 202 may transition to a new state that adjusts at least one or both the sensitivity coefficient or the offset coefficient. This potential change in state is illustrated in FIGS. 3A and 3B within the RUNNING states 302 (e.g., RUNNING NEW, RUNNING STABLE, RUNNING EOL (end of life)). Additionally, upon transitioning to a new RUNNING state 302, the COEFFICIENT states 304 may also subsequently change to account for drift or offset associated with sensor life.

In still other embodiments, it may be preferable to enter different calibration coefficient states 304 based on state input such as the range of analyte concentration being generated by the sensor. For example, in an embodiment where a sensor is configured to measure ketones, it may be preferential to have calibration coefficients associated with a low ketone concentration threshold and a high ketone concentration threshold. It may be beneficial to maintain or utilize calibration coefficients associated with preferred thresholds in embodiments in order to subdivide a nonlinear curve. Splitting up or dividing a nonlinear curve into more than one section, each associated with different calibration coefficients, can enable linearization of the nonlinear curve at preferred concentrations. Additionally, transitioning to states associated with calibration coefficients that are associated with a linearized calibration curve reduces or simplifies processing logic. In still other embodiments, the electronics may “auto-range” as the signal transitions low/high, increasing the current resolution of a potentiostat at lower ranges. Calibration coefficients may optimally adjust to match the changes in either range and/or resolution of the system to improve resolution and accuracy without sacrificing detection range of the sensor. In this non-limiting exemplary illustration, the thresholds for a low and high analyte concentration can enable entry into a state resulting in the use of calibration coefficients associated with low analyte concentration, high analyte concentrations and nominal analyte concentrations.

In other embodiments, a calibration state machine 202 may benefit from an additional layer of initial state that is either derived from or associated with manufacturing data 306 in FIG. 3B. In these embodiments, exemplary manufacturing data may include, but is not limited to information such as lot-specific calibration coefficients. The use of manufacturing data 306 enables an additional number of initial running states that can further adjust or be used to determine the calibration coefficient based on “expected” levels derived from, or provided by the manufacturing data 306.

In some embodiments, unexpected sensor signals or an unexpected change in sensor signals may optionally transition the calibration state machine 202 to a WAITING state. In some embodiments, entering the WAITING state results in no calibration coefficient being applied. Once in the WAITING state, it may be preferable to no longer output sensor data. In many embodiments, the calibration state machine 202 may transition back to a RUNNING state 302 when state inputs meet expected conditions within a specified range or exceed a specified threshold.

Both FIGS. 3A and 3B include an end of life (EOL) state. The calibration state machine 2023 transitions to the EOL state when the state input associated with sensor age exceeds a maximum duration. Once in the EOL state, no further or additional calibration coefficients will be determined until a new sensor is detected. The state inputs discussed above are intended to be exemplary rather than limiting. Additional state inputs may be used or implemented to determine calibration coefficients. The additional state inputs may be associated with an initial state of the calibrations state machine 202. Alternatively, the additional state inputs may be associated with longitudinally state input.

FIG. 4 is an illustration of an exemplary hardware implementation of a calibration state machine 202, in accordance with embodiments of the present invention. Accordingly, the calibration state machine 202 illustrated in FIG. 4 is implemented in logic, memory and feedback necessary to navigate across the various possible states. In many embodiments the calibration state machine 202 includes a clock that determines a current state. For example, the clock or timer may determine the state to be “no change” or “transition to a new state”. As implemented in FIG. 4, the calibration state machine 202 includes multiple programmable logic devices (PLDs) such as configurable logic cells (CLCs). Additionally, multiple calibration state machines may be implemented on a single device wherein each state machine is assigned to one of the multiple sensor channels.

Each state machine PLD array may be configured to select between states based on system and sensor inputs. In preferred embodiments the system and sensor inputs may be both initial and longitudinal. For example, in some embodiments the system and sensor inputs may be instantaneous (e.g., initial) or time-varying (e.g., longitudinal). Moreover, each state machine PLD array may rely on multiple additional logic gates when appropriate. For example, a first stage may check or determine the operational state (e.g., WAITING or RUNNING or EOL) of the system and a secondary stage may select between manufacturing data once the system is in a RUNNING state. Specifically, the secondary stage may not be evaluated or implemented if the operational state is WAITING or EOL. The CURRENT STATE is saved and is checked periodically, the check being triggered by the clock or timer as defined in hardware or software. In many embodiments, the CURRENT STATE is saved in a device register or in a programmable flash memory (PFM)).

Upon detection of the state transition, the calibration state machine may directly access device memory (DMA) to select the appropriate calibration coefficients from a stored coefficient map or table. Selected (CURRENT COEFFICIENTS) are saved in device memory (PFM) for external use by a microcontroller (MCU). In other states such as, but not limited to the system inputs being indicative of input faults such as, but not limited to sensor signal being unstable, out of range, exceeding a noise threshold, or EOL, a state transition may clear the selected calibration coefficients from the PFM to prevent display of invalid calibrated values. In alternative embodiments, rather than clearing the calibration coefficients upon detection of input faults, the calibration state machine may transition to a power down state. When running, the MCU will generate or compute calibrated sensor values by combining longitudinal sensor inputs with the selected calibration coefficients when and if they are available.

In many embodiments, additional features or elements can be included, added or substituted for some or all of the exemplary features described above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. An analyte monitoring system, comprising:
a body worn device including a housing that contains a processor, a memory, a power supply and a sensor interface;
an analyte sensor being coupled to the sensor interface, the analyte sensor having a proximal portion contained within the housing being interfaced with the sensor interface, the analyte sensor further having a distal portion configured to be inserted into a host when positioned outside the housing; and
a finite state machine to select from possible sensor calibration coefficients based on a current state of the analyte monitoring system, an instantaneous input, and time-varying inputs received from the analyte sensor,
wherein the processor generates calibrated sensor values based on the selected calibration coefficients and time-varying inputs from the analyte sensor.

2. The analyte monitoring system of claim 1, wherein the instantaneous input includes a number of analytes being monitored, an analyte or analytes being monitored, and manufacturing information of the analyte sensor.

3. The analyte monitoring system of claim 2, wherein the time-varying inputs include an age of the analyte sensor.

4. The analyte monitoring system of claim 3, wherein the time-varying inputs further include sensor measurement values.

5. The analyte monitoring system of claim 3, wherein a combination of a current state, the instantaneous input and the age of the analyte sensor transition the finite state machine to a new state that selects a different sensor calibration coefficient.

6. The analyte monitoring system of claim 4, wherein a combination of a current state, the instantaneous input and the sensor measurement values transition the finite state machine to a different state based on the sensor measurement values.

7. The analyte monitoring system of claim 6, wherein the different states are based on sensor measurement values being in a low range, medium range, or high range.

8. The analyte monitoring system of claim 2, wherein the manufacturing information enables transition to different states based on expected sensor measurement values.

9. The analyte monitoring system of claim 3, wherein when the sensor age exceeds a maximum duration the finite state machine transitions to an end of life state.

10. The analyte monitoring system of claim 1, wherein the finite state machine is implemented in hardware that includes single write semiconductors.

11. The analyte monitoring system of claim 9, wherein the finite state machine is located within the body worn device.

12. The analyte monitoring system of claim 1, wherein the analyte sensor is configured to monitor a plurality of analytes using a corresponding plurality of channels.

13. The analyte monitoring system of claim 12, wherein each of the corresponding plurality of channels includes a finite state machine to select from possible sensor calibration coefficients.

14. The analyte monitoring system of claim 13, wherein a state of a first finite state machine is used as an input for a second finite state machine.

15. A method to determine calibration coefficients for an analyte monitoring system comprising the operations of:
determining a current state of the analyte monitoring system;
providing an instantaneous input from the analyte monitoring system to a calibration finite state machine;
transitioning the finite state machine to a first state;
receiving time-varying input from the analyte monitoring system, and
transitioning the finite state machine to a second state associated with a first calibration coefficient.

16. The method to determine calibration coefficients for an analyte monitoring system as described in claim 15, further including the operations of:
receiving additional time-varying input from the analyte monitoring system, and
transitioning the finite state machine to a third state associated with a second calibration coefficient.

17. The method to determine calibration coefficients for an analyte monitoring system as described in claim 16, wherein the instantaneous input to the analyte monitoring system includes a number of analytes to be monitored.

18. The method to determine calibration coefficients for an analyte monitoring system as described in claim 17, wherein the instantaneous input of the analyte monitoring system includes the analytes to be monitored.

19. The method to determine calibration coefficients for an analyte monitoring system as described in claim 17, wherein the instantaneous input to the analyte monitoring system includes manufacturing data associated with the analyte monitoring system.

20. The method to determine calibration coefficients for an analyte monitoring system as described in claim 16, wherein the additional time-varying input includes an elapsed time from the transitioning to the first state.
